(19) United States
(12) Patent Application Publication
(10) Pub. No.: US 2023/0094633 A1
(43) Pub. Date: Mar. 30, 2023

(54) Methods and Systems to Determine HLA-DPB1 Expression

(71) Applicant: Laboratory Corporation of America Holdings, Burlington, NC (US)

(72) Inventors: Lakshmanan Krishnan Iyer, Franklin, MA (US); Brian Matthew Norvell, Liberty, NC (US); Jonathan David Williams, Hillsborough, NC (US)

(73) Assignee: Laboratory Corporation of America Holdings, Burlington, NC (US)

(21) Appl. No.: 17/802,913

(22) PCT Filed: Feb. 22, 2021
(86) PCT No.: PCT/US2021/018968

§ 371 Date: Aug. 26, 2022

Related U.S. Application Data
(60) Provisional application No. 62/982,286, filed on Feb. 27, 2020.

(51) Int. Cl. G16B 30/10; C12Q 1/6881; G16H 50/20; G16H 10/40

(52) U.S. Cl. CPC G16B 30/10; C12Q 1/6881; G16H 50/20; G16H 10/40; C12Q 2600/106; C12Q 2600/158; C12Q 2600/156

(57) ABSTRACT

Disclosed are methods and systems and computer program products for determining DPB1 expression without the need for rs9277534 (3′ UTR) sequence data. Such methods are useful in assessing HLA sequencing submitted to or already in donor databases for matches that will reduce the risk of hematopoietic transplant rejection (i.e., graft vs. host disease).

### PRIORITY CLAIM

This application claims the benefit of and priority to U.S. Provisional Application No. 62/982,286, filed on Feb. 27, 2020, which is hereby incorporated by reference in its entirety for all purposes.

### FIELD

The disclosure relates to methods and systems to determine DPB1 expression for HLA typing used to match transplant donors and recipients.

### BACKGROUND

Hematopoietic stem cell transplantation (HSCT) from unrelated donors can cure various blood disorders, however, a high level of donor-recipient HLA compatibility is crucial for success. Currently, matching for HLA-A, -B, -C, -DRB1, DRB3 and -DQB1 alleles is the gold standard. HLA-DPB1 is often considered as well, but it's genetic distance from other HLA genes leads to a frequent mismatch at this locus. However, when mismatched, HLA-DPB1 expression level can play a critical role in hematopoietic stem cell transplantation. Studies have shown donors and recipients who have expression level mismatches for HLA-DPB1 are more likely to develop graft versus host disease (GvHD).

In particular, it has been found that the expression level of HLA-DPB1 can be correlated with an A→G single nucleotide polymorphism (SNP), rs9277534, located in the 3′ untranslated region (UTR) of DPB1 (Thomas et al., J. Virol., 86:6979-85 (2012)). The “A” allele is associated with weak DPB1 expression and the “G” allele is associated with strong DPB1 expression (Petersdorf et al., New Engl. J. Med. 373:599-609 (2015)). Studies indicate that the rs9277534 A→G polymorphism is tightly linked to variants at 7 specific nucleotides in DPB1 exon 3 (See e.g., Schone et al., Human Immunol., 79:20-27 (2018)).

The risk of GvHD associated with HLA-DPB1 mismatching is influenced by the HLA-DPB1 rs9277534 expression marker. For recipients of HLA-DPB1 mismatched transplants (e.g., exon 2 mismatching or other mismatches) from donors with the low-expression allele (rs277534A), recipients having the high strong expression allele will have a high risk of acute GvHD (Petersdorf et al., (2015)). However, the 3′ UTR containing rs9277534 is currently not covered by routine genotyping assays for HLA-DPB1 and expression assays are not routinely performed.

Thus, there is a need to develop methods to characterize DPB1 expression for HSCT transplant patients for samples that have been sequenced as a means to identify appropriate donor: recipient pairs.

### SUMMARY

Embodiments of the disclosure include methods and systems for analyzing long read sequence data to predict DPB1 expression level. In certain embodiments, the methods and systems are computer implemented. Also disclosed are computer program products for implementation of the methods and/or systems of the disclosure. In an embodiment, the disclosed methods and systems are useful for determining HSCT donor-recipient compatibility. The methods and systems do not require determination of the sequence of rs9277534 or measurement of the DPB1 expression as mRNA or protein levels.

For example, in one embodiment, a computer-implemented method is provided for analyzing sequence data from a subject to predict DPB1 expression level in the subject comprising the computer-implemented steps of:

(a) obtaining, using a long read sequencer, a query nucleic acid sequence from a sample of the subject;

(b) aligning, using a computer-implemented alignment program, the query nucleic acid sequence from the subject to a reference nucleic acid sequence, the reference and query nucleic acid sequences comprising long read sequence data for at least exon 3 of DPB1; and

(b) identifying, using a computer-implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises the following steps:
(i) comparing nucleotides within the aligned query nucleic acid sequence and the reference nucleic acid sequence to identify differences between the query nucleic acid sequence and the reference nucleic acid sequence;
(ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1;
(iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and
(iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.

In some embodiments, a system is provided that includes one or more data processors and a non-transitory computer readable storage medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present disclosure has been specifically described by embodiments and optional features, modification and variation of the concepts herein disclosed may be resorted to by those skilled in the art, and that such modifications and variations are considered to be within the scope of this disclosure as defined by the appended claims.

### BRIEF DESCRIPTION OF THE FIGURES

The disclosure may be better understood by the following non-limiting figures.

FIG. 1 shows sequences in the DPB1 cDNA that are associated with the rs9277534 A allele and weak expression and the rs9277534 G allele and strong expression in accordance with embodiments of the disclosure.

FIG. 2 shows nucleotide variations DPB1 cDNA for haplotype 01:01:01:01 (SEQ ID NO: 1) that are associated with the rs9277534 G allele and strong expression and sequences in the DPB1 cDNA for haplotype 02:01:02:01 (SEQ ID NO: 2) that are associated with the rs9277534 A allele and weak expression.

FIG. 3 shows use of the disclosed method to identify exon 3 motifs in accordance with various embodiments of the disclosure. The query positive strand (Qry+) sequence (SEQ ID NO: 4) has a sequence of the strong expression motif (solid rectangles) at exon 3 positions 20, 27, 52, 87, 234, 242 and 270 as well as other variations (dotted rectangles) and the reference positive strand sequence (Ref+) (SEQ ID NO: 3) has the weak expression motif. In this figure, the sequencing alignment starts at the fourth nucleotide for exon 3.

FIG. 4 shows use of the disclosed algorithm to identify exon 3 motifs in accordance with various embodiments of the disclosure. The query positive strand (Qry+) (SEQ ID NO: 5) sequence has a sequence of the weak expression motif (solid rectangles) at positions 20, 27, 52, 87, 234, 242 and 270 as well as other variations (dotted rectangles). The reference positive strand sequence (Ref+) (SEQ ID NO: 3) is the same reference sequence of FIG. 3, and also has the weak expression motif. In this figure, the sequencing alignment starts at the fourth nucleotide for exon 3.

FIG. 5 shows an embodiment of a method of the disclosure for determining DPB1 exon 3 motifs associated with strong and weak expression.

FIG. 6 shows another embodiment of a method of the disclosure for determining DPB1 exon 3 motifs associated with strong and weak expression.

FIG. 7 shows a comparison of the DPB1 34:01 (weak expression and the Ref+ sequence from FIG. 3) (SEQ ID NO: 3) and the DPB1 01:01 (strong expression) (SEQ ID NO: 6) sequences for exon 3 in accordance with an embodiment of the disclosure. It can be seen that only exon 3 positions 7, 20, 27, 52, 87, 234, 242, 270 show differences between the reference (Ref+) and the query (Qry+). Concise Idiosyncratic Gapped Alignment Report (CIGAR) presented at the top of figure denotes how this alignment would be reported in the algorithm for further analysis.

FIG. 8 shows a comparison of the DPB1 34:01 (weak expression) (SEQ ID NO: 3) and the DPB1 02:01 (weak expression) (SEQ ID NO: 7) sequences for exon 3 in accordance with an embodiment of the disclosure. It can be seen that for exon 3 positions 7, 20, 27, 52, 87, 234, 242, 270 (and other exon positions) there is an exact match. CIGAR string on top of figure denotes how this alignment would be reported in the algorithm for further analysis.

FIG. 9 shows output as a CIGAR for various sequences analyzed using the method in accordance with various embodiments of the disclosure showing five strong sequences and five weak sequences; one of the weak sequences has variant at position 243 of DPB1 exon 3.

FIG. 10 shows the sequence alignment as compared to a reference (Ref+) (SEQ ID NO:3) for the cs:z::242*ct:59 weak sequence of FIG. 9 (SEQ ID NO: 8) in accordance with an embodiment of the disclosure exhibiting the variant at position 243.

FIG. 11 shows an exemplary computing device in accordance with various embodiments of the disclosure.

FIG. 12 shows additional results for the methods and systems in accordance with various embodiments of the disclosure obtained using an RSII SMRTcell.

FIG. 13 shows additional results for the methods and systems in accordance with various embodiments of the disclosure obtained using a Sequel SMRTcell. A total of 299 motifs in 88 samples where obtained.

FIG. 14 shows a visual display of the results for HLA genes DRB1, DRB3, DQB1 and DPB1 in accordance with an embodiment of the disclosure. The 02:01:026 sequence with 133 reads is characterized as weak, and the 0:3:01:01G sequence with 383 reads is characterized as strong with 7 mismatches at the defined positions in exon 3 that correlate to the strong expression SNP.

FIG. 15 shows a total of 31,274 DPB1 sequences were analyzed and of those, 10,774 were typed as “Strong,” 20,369 were typed as “Weak,” and 131 were typed as “Undetermined” in accordance with an embodiment of the disclosure.

FIG. 16 shows a plot of the positions in exon 3 where unique variants to a weak reference occur in accordance with an embodiment of the disclosure.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### Definitions

In order for the disclosure to be more readily understood, certain terms are first defined. Additional definitions for the following terms and other terms are set forth throughout the specification.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “and/or” generally is used to refer to at least one or the other. In some case the term “and/or” is used interchangeably with the term “or.” The term “including” is used herein to mean, and is used interchangeably with, the phrase “including but not limited to.” The term “such as” is used herein to mean, and is used interchangeably with, the phrase “such as but not limited to.”

Unless defined otherwise, all technical and scientific terms used herein have the same meaning as commonly understood by one of ordinary skill in the art.

Also as used herein, “at least one” contemplates any number from 1 to the entire group. For example, for a listing of four variants, the phrase at “least one” is understood to mean 1, 2, 3 or 4 variants. Similarly, for a listing of 10 variants, the phrase “at least five” is understood to mean 5, 6, 7, 8, 9, or 10 variants.

Also, as used herein, “comprising” includes embodiments more particularly defined using the term “consisting of.”

Also, as used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

Also, as used herein, when an action is “based on” something, this means the action is based at least in part on at least a part of the something.

Activity: As used herein, the term “activity” refers to the expression levels of a gene. For example, DPB1 activity refers to the levels of DPB1 mRNA and/or protein.

Allele: As used herein, the term “allele” refers to different versions of a nucleotide sequence of a same genetic locus (e.g., a gene).

Coding Sequence: As used herein, the term “coding sequence” refers to a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the encoding sequence can be deduced therefrom. As used herein, the term “non-coding sequence” refers to a sequence of a nucleic acid or its complement, or a part thereof, that is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

Contig: As used herein the term “contig” refers to a DNA sequence that represent a consensus sequence for a region of DNA assembled from a set of identical full or partially overlapping DNA sequencing reads. In some cases the sequencing reads are produced from a Next Generation Sequencing reaction.

Deletion: As used herein, the term “deletion” encompasses a mutation that removes one or more nucleotides from a naturally-occurring nucleic acid.

Exon: As used herein the term “exon” refers to a nucleic acid sequence that is found in mature or processed RNA after other portions of the RNA (e.g., intervening regions known as introns) have been removed by RNA splicing. As such, exon sequences generally encode for proteins or portions of proteins. An intron is the portion of the RNA that is removed from surrounding exon sequences by RNA splicing.

Expression and expressed RNA: As used herein expressed RNA is an RNA that encodes for a protein or polypeptide (“coding RNA”), and any other RNA that is transcribed but not translated (“non-coding RNA”). The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both. As used herein, DPB1 “strong” expression comprises expression levels on the order of 1.5 to 2 times greater than weak expression (see e.g., Petersdorf et al., (2015).

Gene: As used herein the term “gene” refers to a unit of heredity. Generally, a gene is a portion of DNA that encodes a protein or a functional RNA. A gene is a locatable region of genomic sequence corresponding to a unit of inheritance. A gene may be associated with regulatory regions, transcribed regions, and or other functional sequence regions.

Genotype: As used herein, the term “genotype” refers to the genetic constitution of an organism. More specifically, the term refers to the identity of alleles present in an individual. “Genotyping” of an individual or a DNA sample refers to identifying the nature, in terms of nucleotide base, of the two alleles possessed by an individual at a known polymorphic site.

Heterozygous: As used herein, the term “heterozygous” refers to an individual possessing two different alleles of the same gene. As used herein, the term “heterozygous” encompasses “compound heterozygous” or “compound heterozygous mutant.” As used herein, the term “compound heterozygous” refers to an individual possessing two different alleles. As used herein, the term “compound heterozygous mutant” refers to an individual possessing two different copies of an allele, such alleles are characterized as mutant forms of a gene.

Homozygous: As used herein, the term “homozygous” refers to an individual possessing two copies of the same allele. As used herein, the term “homozygous mutant” refers to an individual possessing two copies of the same allele, such allele being characterized as the mutant form of a gene.

Insertion or addition: As used herein, the term “insertion” or “addition” refers to a change in an amino acid or nucleotide sequence resulting in the addition of one or more amino acid residues or nucleotides, respectively, as compared to the naturally occurring molecule.

Long read sequencing: As used herein, “long read sequencing”, also called third-generation sequencing, is a DNA sequencing technique which can determine the nucleotide sequence of long read sequences of DNA between 10,000 and 100,000 base pairs at a time. This removes the need to cut up and then amplify DNA which is normally required in other DNA sequencing techniques. Long read sequencing also allows unambiguous linkage between exon 2 and 3 of DPB1. Short read sequencing frequently loses phasing and inability to link the strong or weak motif to the appropriate exon 2.

Long read sequence: As used herein a “long read sequence” is a continuous read of a single molecule, such as a PCR amplicon, that allows the elimination of phasing needed in shorter read technologies.

Mutation and/or variant: As used herein, the terms “mutation” and “variant” are used interchangeably to describe a nucleic acid or protein sequence change. The term “mutant” as used herein refers to a mutated, or potentially non-functional form of a gene. The term includes any mutation that renders a gene not functional from a point mutation to large chromosomal rearrangements as is known in the art.

Nucleic acid: As used herein, the term “nucleic acid” refers to a polynucleotide such as deoxyribonucleic acid (DNA) or ribonucleic acid (RNA). The term is used to include single-stranded nucleic acids, double-stranded nucleic acids, mRNA, and RNA and DNA made from nucleotide or nucleoside analogues.

Polymorphism: As used herein, the term “polymorphism” refers to the coexistence of more than one form of a gene or portion thereof.

Query sequence: As used herein, the term “query sequence” or “query” or “Qry” refers to an uncharacterized nucleic acid consensus sequence being compared against a known sequence for analysis of the unknown sequence content. The sequence may be deoxyribonucleic acid such as genomic DNA or copy DNA amplified from messenger RNA (cDNA) or ribonucleic acid (RNA). In an embodiment, the sequence is genomic DNA.

Reference sequence: As used herein, the term “reference sequence” or “reference” or “Ref” refers to a known sequence that is used as a standard for analysis of a query, or unknown, sequence. The sequence may be deoxyribonucleic acid such as genomic DNA or copy DNA amplified from messenger RNA (cDNA) or ribonucleic acid (RNA). In an embodiment, the sequence is genomic DNA.

Sample: As used herein, the term “sample” refers to any type of suitable biological specimen or sample (e.g., a test sample) that nucleic acid can be isolated. A biological specimen or sample can be any specimen or sample that is isolated or obtained from a subject or part thereof (e.g., a human subject, a pregnant female, a fetus). Non-limiting examples of a specimen or sample include fluid or tissue from a subject, including, without limitation, blood or a blood product (e.g., serum, plasma, or the like), umbilical cord blood, chorionic amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo), celocentesis sample, cells (blood cells, placental cells, embryo or fetal cells, fetal nucleated cells or fetal cellular remnants) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof.

Sense strand vs. anti-sense strand: As used herein, the term “sense strand” refers to the strand of double-stranded DNA (dsDNA) that includes at least a portion of a coding sequence of a functional protein. As used herein, the term “anti-sense strand” refers to the strand of dsDNA that is the reverse complement of the sense strand. As used herein, the positive or +strand is the sense strand. As used herein, “+” refers to the sense strand and “−” refers to the anti-sense strand.

Subject or Individual or Patient: As used herein, the term “subject” or “individual” refers to a human or any non-human animal. A subject or individual can be a patient, which refers to a human presenting to a medical provider for diagnosis or treatment of a disease, and in some cases, wherein the disease requires a hematopoietic stem cell transplant. Also, as used herein, the terms “individual,” “subject” or “patient” includes all warm-blooded animals.

### Methods for Analyzing Sequence Data to Predict DPB1 Expression Level

HLA-DPB1 expression has been shown to play a critical role in hematopoietic stem cell transplantation. Studies have demonstrated donors and recipients who have expression level mismatches for HLA-DPB1 are more likely to develop graft versus host disease. Embodiments of the disclosure include methods for analyzing sequence data to predict DPB1 expression level. The method may be used to evaluate a donor database to find matches for individuals in need of a transplant or during sequencing for HLA typing in a reference lab. In an embodiment, the transplant is a hematopoietic stem cell transplant. In certain embodiments, at least some of the steps may be computer-implemented.

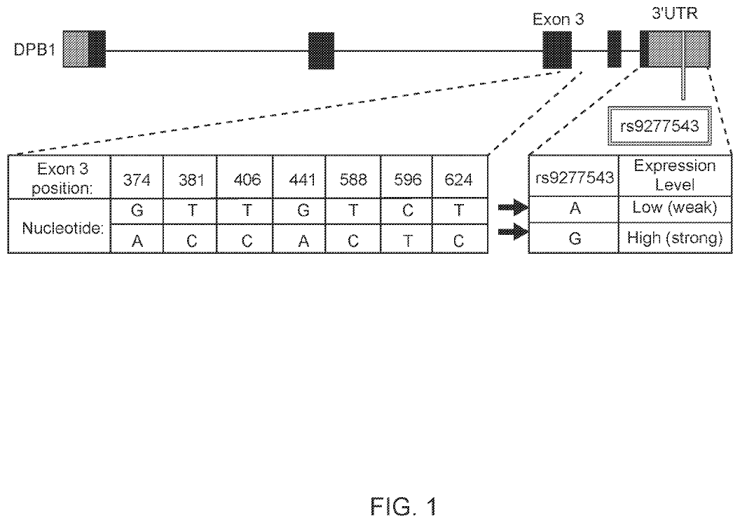

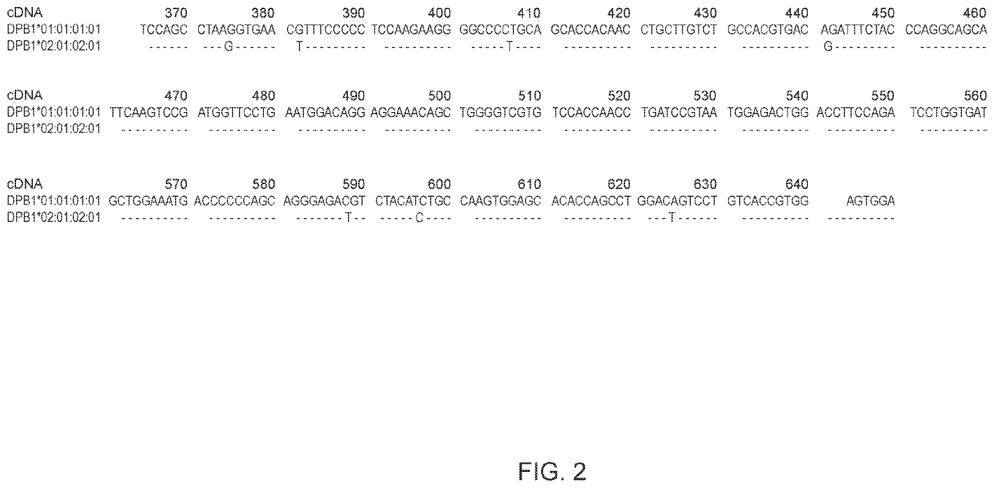

The methods can solve a need to determine the expression levels of DPB1 as part of an HLA haplotyping analysis. Often the analysis of DPB1 sequences for HLA haplotyping is based only on DPB1 exon 2, and so does not include the sequences that are outside of exon 2 (i.e., exons 1, 3, 4 and 5, upstream and/or downstream sequences, regulatory sequences, epigenetic sequences, introns and the like). However, there is no indication that polymorphisms in exon 2 are linked to DPB1 activity. Instead, it has been discovered that the marker rs9277534 in DPB1′s 3′ UTR genetically controls the HLA-DPB1 expression, and seven specific base pairs within exon 3 can predict the single nucleotide variant of the marker rs9277534 (the single nucleotide variant determinative of expression level). Therefore, analyzing these seven well defined locations within exon 3 using sequence data obtained by long read sequencing can predict HLA-DPB1 expression. FIG. 1 shows the DPB1 gene structure with locations and bases in exon 3 that predict the rs9277543 marker. The single nucleotide variant at this marker controls DPB1 expression levels. FIG. 2 shows the Exon 3 sequence for DPB1*04:01:01:01 showing weak expression sequence and DPB1*01:01:01:01 showing strong expression sequence.

In various embodiments, a method is provided for that comprises determining the sequence of DPB1 exon 3, and in particular, nucleotide variations that are linked to DPB1 activity. In one embodiment, disclosed are methods for analyzing the seven well defined locations within DPB1 exon 3 that are linked to DPB1 activity. The sequence may be a contig that is constructed as part of a long read or short read sequencing experiment. In some embodiments, the sequencing is performed by next generation sequencing (NGS) such as long read sequencing. Thus in certain embodiments, the methods may be used to predict HLA-DPB1 expression. The methods may also be used to predict transplant rejection and/or graft vs host disease (GvHD). In certain embodiments, the methods and systems use bioinformatics to analyze exon 3 sequences for donor and patient NGS data (e.g., long read sequences) to predict the expression level for each HLA-DPB1 allele.

For example, in an embodiment a method is provided for analyzing long read sequence data from a subject to predict DPB1 expression level in the subject, the method comprising the steps of:

(a) obtaining, using a long read sequencer, a query nucleic acid sequence from a sample of the subject;

(b) aligning, using a computer-implemented alignment program, the query nucleic acid sequence from the subject to a reference nucleic acid sequence, the reference and query nucleic acid sequences comprising long read sequence data for at least exon 3 of DPB1; and

(c) identifying, using a computer-implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises the following steps:
(i) comparing nucleotides within the aligned query nucleic acid sequence and the reference nucleic acid sequence to identify differences between the query nucleic acid sequence and the reference nucleic acid sequence;
(ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1;
(iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and
(iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.

In an embodiment, the method further comprises the step of determining the query sequence for the subject. For example, the query sequence may be obtained from long read sequence data generated (prior to step (b)) as part of a next generation sequencing experiment such as long read sequencing. Or another method of nucleic acid sequencing may be used.

In various embodiments, at least some of the steps of the method are computer-implemented. For example, in an embodiment, step (a), (b), (c), or any combination thereof are computer-implemented. In certain embodiments, the steps of the method are implemented on using a long read sequencer, using a computer-implemented alignment program, and using a computer-implemented algorithm, as described herein in detail. In some instances, the long read sequencer may include the computer-implemented alignment program and/or the computer-implemented algorithm. In other instances, the long read sequencer is a separate from the computer-implemented alignment program and/or the computer-implemented algorithm, and the computer-implemented alignment program and/or the computer-implemented algorithm are implemented in one or more specialized computing devices.

In an embodiment, the method further comprises identifying individual alleles for the DPB1 expression motif and/or other sites of interest for the subject.

The sequence data may include data in addition to the sequence for exon 3 of DPB1. In an embodiment, the sequence data is long read sequence data. The data may encompass genomic sequencing for the DPB1 gene. The data may further comprise genomic sequencing for the DRB1, DRB3 and/or DQB1 genes. In an embodiment, the sequence data does not include the 3′ UTR for DPB1. Also in an embodiment, the sequence data does not include the sequence of rs9277534. In an embodiment, the data is sequence data from any bone marrow registry or data generated to submit to a registry or transplant center.

The alignment may be performed by a variety of different methods. In an embodiment, and used in the examples herein, the alignment is carried out using the program Minimap2 (Heng Li, Bioinformatics, 34(18), 2018, 3094-3100). Minimap2 is implemented in the C programming language and comes with APIs in both C and Python. It is distributed under the MIT license, free to both commercial and academic uses. As is known in the art, Minimap2 follows a seed-chain-align procedure typical of most full-genome aligners. Essentially the program collects minimizers of the reference sequences and indexes the minimizers in a hash table. Then for each query sequence, using the hash of a minimizer and the value being a list of locations, minimap2 takes query minimizers as seeds, finds exact matches (i.e., anchors) to the reference, and identifies sets of co-linear anchors as chains. For base-level alignment, minimap2 can apply dynamic programming (DP) to extend from the ends of chains and to close regions between adjacent anchors in chains (see e.g., Heng Li, Bioinformatics, 34(18), 2018, 3094-3100) to produce alignments. Other methods that may be used include BLASR (v1.MC.rc64; Chaisson and Tesler, 2012), BWA-MEM (v0.7.15; Li, 2013), GraphMap (v0.5.2; Sović et al., 2016), Kart (v2.2.5; Lin and Hsu, 2017), minialign (v0.5.3 available on the web at github.com) and NGMLR (v0.2.5; Sedlazeck et al., 2018).

In an embodiment, the disclosed method may further comprise defining the weak expression motif as comprising the following: a G at position 20 of exon 3, a T at position 27 of exon 3, a T at position 52 of exon 3, a G at position 87 of exon 3, a T at position 234 of exon 3, a C at position 242 of exon 3, and a T at position 270 of exon 3. Additionally and/or alternatively, the disclosed method may further comprise defining the strong expression motif as comprising the following: an A at position 20 of exon 3, a C at position 27 of exon 3, a C at position 52 of exon 3, an A at position 87 of exon 3, a C at position 234 of exon 3, a T at position 242 of exon 3, and a C at position 270 of exon 3. The method may further comprising determining if the strong expression motif is linked to the rs9277534 G allele and/or the weak expression motif is linked to the rs9277534 A allele. In other embodiments, if the nucleotides at exon 3 positions 20, 27, 52, 87, 234, 242 and 270 are not characteristic of either the weak expression motif or the strong expression motif, the disclosed method may comprise defining the allele as indeterminate.

The disclosed method may further include determining any differences between the query sequence and the reference sequence at other positions of DPB1 exon 3. For example, the reference and/or query sequence may comprise sequence data for the entire DPB1 gene. Additionally and/or alternatively, the disclosed method may further comprise determining any other differences between the query sequence and the reference sequence (e.g., in other exons of DPB1 and/or other HLA genes of interest such as HLA-A, -B, -C, DRB1, DRB3 and/or DQB1). For example, the reference and query sequence may further comprise sequence data for at least one of DRB1, DRB3 and DQB1. Also, the method may include determining if other variants in DPB1 exon 3, and/or other regions of the DPB1 gene are linked to the rs9277534 G allele associated with strong expression and/or the rs9277534 A allele associated with weak expression. In an embodiment, this additional data may be submitted to a caregiver or a transplant database. In this way, additional variants associated with DPB1 expression can be assessed for use as indicators of DPB1 expression levels.

In an embodiment, the additional sequence data is compiled with the sequence of DPB1 exon 3. For example, linkage of other sequences with the activity profile of DPB1 exon 3 may be useful in further characterizing the basis for GvHD in transplant recipients.

In some embodiments, if the number of differences between the query sequence and the reference sequence at positions other than positions 20, 27, 52, 87, 234, 242 and 270 in DPB1 exon 3 is greater than a predetermined number, (e.g., ten), the query sequence is excluded from further analysis. For example, this may be the case when the sequence being analyzed is not actually DPB1 but another sequence present in the NGS data.

In certain embodiments, the subject is a potential donor for a transplant recipient. In an embodiment, the recipient is a hematopoietic stem cell transplant (HSCT) recipient. The analysis may be used to match potential donors to transplant recipients. Thus, in certain embodiments, the method may further comprise providing the results to a caregiver to reduce the risk of graft vs host disease in a transplant recipient. Or, the method may further comprise providing the sequence analysis to a database for future distribution to a caregiver and/or potential recipient. In certain embodiments, the recipient is a hematopoietic stem cell transplant (HSCT) recipient.

Thus, the disclosed methods may comprise determining if the query exhibits the sequence characteristic of a weak expression motif or a strong expression motif or neither, thereby assessing the expression level of the subject's DPB1. FIG. 1 shows a comparison of nucleotides at specific positions in DPB1 cDNA. By subtracting the length of exons 1 and 2 (354 nt) the position for each of these nucleotides in exon 3 is described (i.e., 20, 26, 52, 87, 234, 242 and 270). FIG. 1 shows the sequence motifs associated with the rs9277534 A allele (weak expression) and or rs9277534 G allele (strong expression). For example, FIG. 2 shows a comparison of exon 2 sequences for the HLA haplotype 01:01:01:01 having the DPB1 strong expression exon 3 motif for DPB1 as compared to the HLA haplotype 02:01:02:01 having the DPB1 weak expression exon 3 motif for DPB1. Numbering is based on the cDNA sequence.

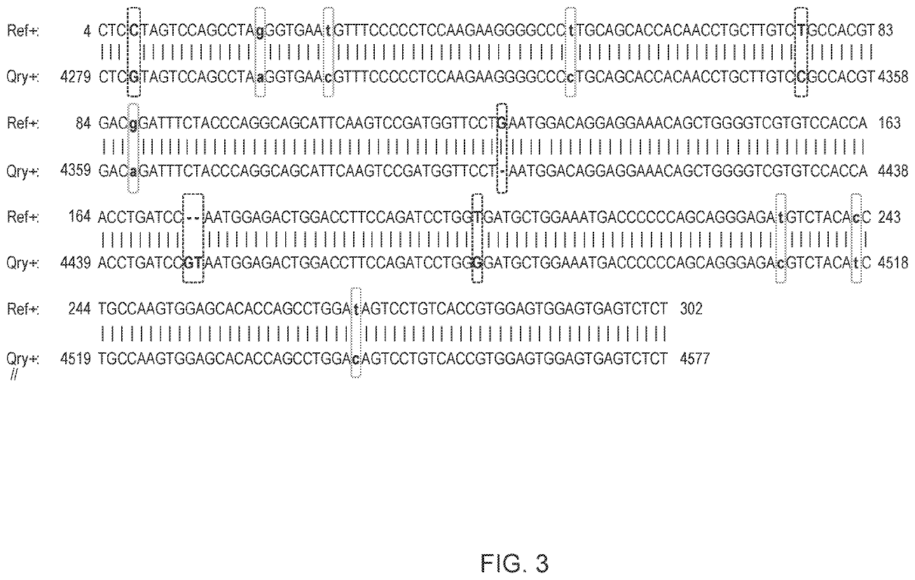

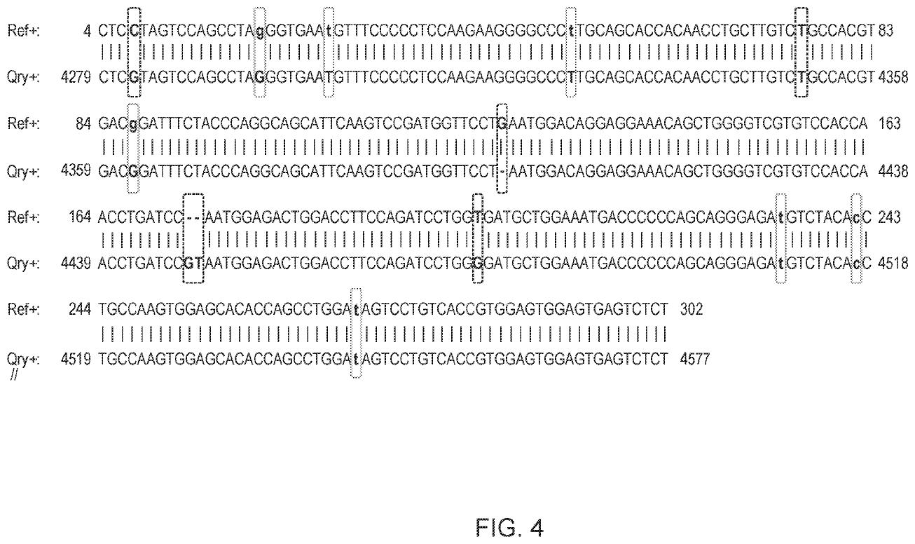

FIGS. 3 and 4 show embodiments for the comparison of sequence data from two different query samples (Qry) to a Reference sequence (Ref) encoding for the weak genotype. In the figures, the + sign denotes the positive (sense or coding) strand (i.e., the strand corresponding directly to the sequence of the mRNA transcript). Thus, FIG. 3 shows use of the disclosed algorithm to identify strong exon 3 motifs; the query (Qry+) sequence has a sequence of the strong expression motif (solid line rectangles) at positions 20, 27, 52, 87, 234, 242 and 270 as well as other variants (dashed line rectangles). In this experiment, the reference sequence (Ref+) has the weak expression motif. FIG. 4 shows use of the disclosed algorithm to identify weak exon 3 motifs; the query (Qry+) sequence has a sequence of the weak expression motif (solid line rectangles) at positions 20, 27, 52, 87, 234, 242 and 270 as well as other variations (dashed line rectangles). In this figure, the sequencing starts at the fourth nucleotide for exon 3. In this experiment, the reference sequence (Ref+) has the weak expression motif.

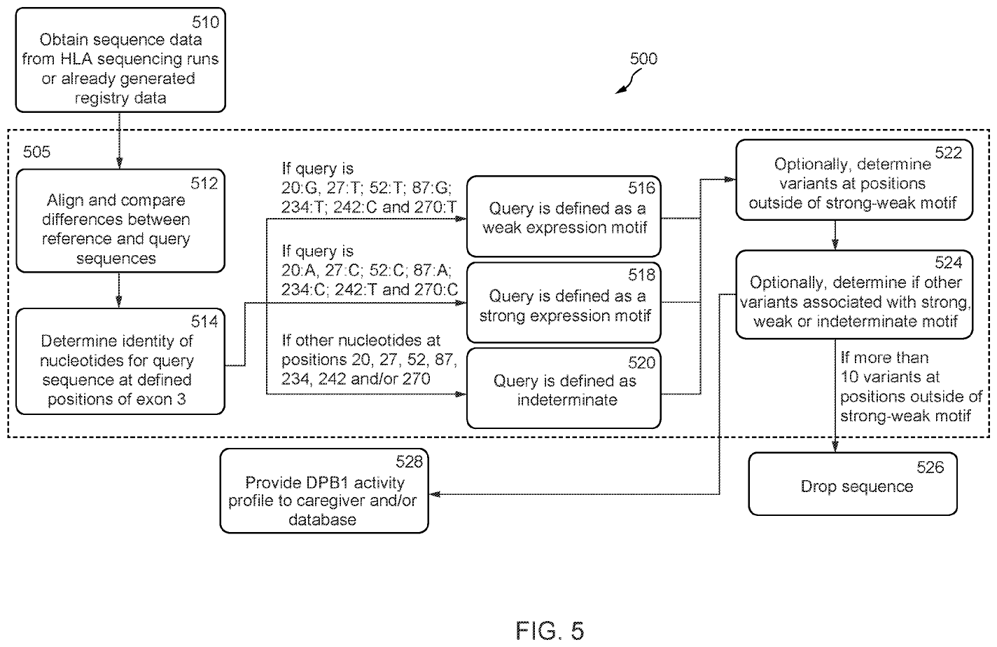

FIG. 5 depicts an embodiment of a computer-implemented and/or algorithmic method 500 that can be used to analyze long sequence read data to predict DPB1 expression. The method may comprise the step 510 of obtaining sequence data from a database or HLA sequencing run. In an embodiment, a query nucleic acid sequence is obtained, using a long read sequencer, from a sample of the subject. Examples of a long read sequencer that could be used in accordance with various embodiments include the Pacific Biosciences RSII, Sequel, and Sequel II, as well as Oxford Nanopore. The long read sequencing data is more applicable to clinical samples for DBI expression because it allows unambiguous linkage between exon 2 and 3 of DPB1 and minimizes phasing loss and the inability to link the strong or weak motif to the appropriate exon 2. In an embodiment, the database is any bone marrow registry database used for selection of donors based on HLA typings. In an embodiment. the query nucleic acid sequences comprise long read sequence data for at least exon 3 of DPB1. Additionally, other sequence data may be included (e.g., other exons of DPB1 and/or other HLA genes of interest such as HLA-A, -B, -C, DRB1, DRB3, and/or DQB1).

The method may include the step 512 of aligning the reference and query sequences and describing and/or comparing differences between the two sequences. In an embodiment, both the reference and query sequences comprise at least exon 3 of DPB1. The method may also include the step 514 of determining the identity of the nucleotides for the query sequence at defined positions in exon 3 of DPB1.

Next the method may comprise the steps of determining if the query exhibits the sequence characteristic of a weak expression motif or a strong expression motif or neither, thereby assessing the expression level of the subject's DPB1. Thus, if the query has the sequence of: a G at position 20 of exon 3, a T at position 27 of exon 3, a T at position 52 of exon 3, a G at position 87 of exon 3, a T at position 234 of exon 3, a C at position 242 of exon 3, and a T at position 270 of exon 3, it is defined as a weak expression motif 516. If the query has the sequence of: an A at position 20 of exon 3, a C at position 27 of exon 3, a C at position 52 of exon 3, an A at position 87 of exon 3, a C at position 234 of exon 3, a T at position 242 of exon 3, and a C at position 270 of exon 3 it is defined as a strong expression motif 518. If, however, the nucleotides at exon 3 positions 20, 27, 52, 87, 234, 242 and 270 are not characteristic of either the weak expression motif or the strong expression motif the method may comprise defining the allele as indeterminate 520.

The method may include the optional steps of determining if there are variants in the query (as compared to the reference) at other positions in DPB1 exon 3, or other exons of DPB1 and/or other sequences important for transplant compatibility 522. The method may include determining if any of the other variants are linked to any of the weak, strong or indeterminate motifs 524. The method may further comprise determining if the strong expression motif is linked to the rs9277534 G allele or the weak expression motif is linked to the rs9277534 A allele (not shown in FIG. 5).

In the case the number of differences between the query sequence and the reference sequence at positions other than exon 3 positions 20, 27, 52, 87, 234, 242 and 270 in DPB1 exon 3 is greater than a pre-defined cut-off (e.g., 5, or 7, or 8, or 10, or 15, or 20, or 25, or 30, or greater), the query sequence may be excluded from further analysis 526. For example, this may be the case when the sequence being analyzed is not actually DPB1 but another sequence present in the sequence data.

The method may include a final step of outputting a result of the analysis. The result is a prediction of a DPB1 expression, which may be used in downstream processing or analysis to match potential donors to transplant recipients. Thus, in certain embodiments, the method may further comprising providing the results to a database or to a caregiver 528 to reduce the risk of graft vs host disease in a transplant recipient. In certain embodiments, the recipient is a hematopoietic stem cell transplant (HSCT) recipient. In certain embodiments, the method may further comprise matching one or more potential donors to one or more transplant recipients using the results of the analysis. Advantageously, the techniques described herein used to analyze long sequence read data to ultimately predict DPB1 expression reduce computing time, provide a more accurate prediction of DPB1 expression, and reduce clinical risk caused by mistakes in calculating/analyzing the long sequence read data.

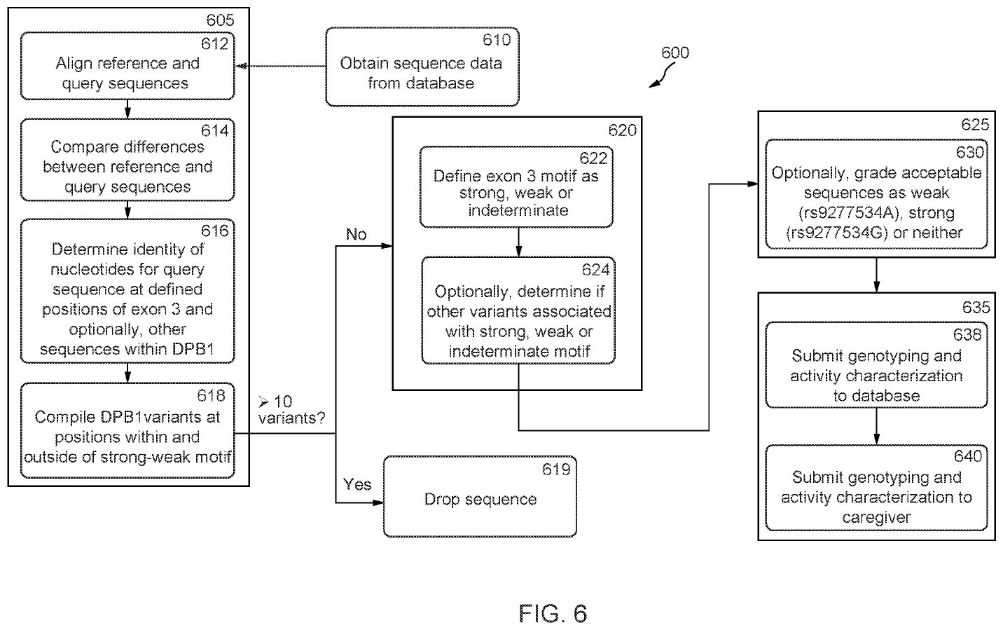

FIG. 6 shows an example of another embodiment of a method 600 of the disclosure. Thus, as depicted in FIG. 6, these steps may comprise obtaining sequence data from a database or HLA sequencing run before submission to a database 610. In an embodiment, a query nucleic acid sequence 605 is obtained, using a long read sequencer, from a sample of the subject. In an embodiment, the database is any bone marrow registry database used for selection of donors based on HLA typings. The next few steps may comprise characterization of the query sequence 605. Thus, the method may comprise the step 612 of aligning the reference and query sequences. Next, the method may comprise comparing the differences between the reference and query sequences 614. The method may further include the step 616 of determining the identity for the query sequence at defined positions of DPB1 exon 3 and optionally, other sequences e.g., in other exons of DPB1 and/or other HLA genes of interest such as HLA-A, -B, -C, DRB1, DRB3 and/or DQB1. The method may also comprise the step 618 of compiling the variants in the query sequence within and optionally outside of the strong-weak motif of exon 3. If there is more than a predetermined number of variants (e.g., 10) the sequence may be “dropped” from the analysis 619. In some embodiments, this results in the sequence not being considered as predicting whether the donor is a suitable donor for the recipient in question.

If, however, there are less than 10 variants outside of the strong-weak motif position, the sequence may be further analyzed 620. This may include identifying the query sequence as having an exon 3 motif that is either weak, strong or indeterminate 622. In an embodiment, this determination based on the sequence at exon 3 positions 20, 27, 52, 87, 234, 242 and 270 as described herein. Also, the analysis may determine if other variants within exon 3 or other DPB1 regions are associated with (i.e., display genetic linkage to) either the weak or strong (or indeterminate) motif 624.

At this point further analysis may be performed 625. For example, the sequence of the exon 3 motif (i.e., strong, weak or indeterminate) may be linked to the sequence within rs9277534, wherein the “A” allele is postulated to be associated with “weak” expression and the “G” allele is postulated to be associated with strong expression. Thus, the data may be used to confirm the linkage of rs9277534 A/G with weak and/or strong expression, respectively, and/or to further refine the genotypic analysis (i.e., by finding other variants associated with the rs9277534 A and G alleles, respectively) 630.

Finally, the results may be submitted to a third party 635. For example, the results may be added to a database 638 and/or provided to a care-giver 640. In an embodiment, the database may be the same database from which the query (and/or reference) sequence was obtained. Or, the database may be a different database. In some embodiments, the care-giver may be a physician hoping to find a donor match for a particular recipient.

Also disclosed herein are methods for reporting the analysis. In an embodiment, a Concise Idiosyncratic Gapped Alignment Report (CIGAR) approach is used. In this analysis the sequence is analyzed 5′ to 3′ using a predetermined start site. Variants are scored based on their nucleotide identity (e.g., A, C, G or T), the nature of the variation (insertion, deletion, base change) and the position of the variant within a region of the sequence (e.g., contig). For example, as discussed above, a strong exon 3 motif for a query sequence could be reported based on the nucleotide identities at positions 20, 27, 52, 87, 234, 242 and 270 of the exon. Or the variants may be identified based on their position in the cDNA, or the genome. Such a representation can be based on the absolute identity of the nucleotides at these positions or as any changes compared to a reference sequence. Thus, the sequence shown in FIG. 4 for a weak motif may be represented as having the following sequence at the positions of interest: 20:g, 27:t, 52:t, 87:g, 234:t, 242:c and 270:t (i.e., a non-CIGAR approach). Alternatively, where a weak motif sequence is the reference, the sequence may be represented as: 20:gg, 27:tt, 52:tt, 87:gg, 234:tt, 242:cc and 270:tt, to indicate there are no variants at the motif positions of exon 3 in the reference (first nucleotide of the pair) as compared to the query (second nucleotide of the pair). Similarly, the strong sequence in FIG. 3 may be denoted as: 20:ga, 27:tc, 52:tc, 87:ga, 234:tc, 242:ct and 270:tc (non-CIGAR approaches).

However, using the CIGAR method, the motif may be reported as any changes between a reference and a query sequence independent of the actual numbering used to identify the position of the nucleotide within a specific exon, cDNA and/or genomic sequence. This can facilitate rapid comparison of contigs of sequence data. In an embodiment, the cs CIGAR tag encodes differences in sequences in the short form or the entire query and reference sequences in the long form (which is the form for which reference sequence data is generally provided).

In an embodiment, the scoring method is as outlined in Table 1.

TABLE 1

| Operation | Regular Expression | Description |
|---|---|---|
| = | [ACGTN]+ | Identical sequence (long form) |
| : | [0-9]+ | Identical sequence length |
| * | [acgtn][acgtn] | Substitution: reference to query |
| + | [acgtn]+ | Insertion to the reference |
| − | [acgtn]+ | Deletion from the reference |
| ~ | [acgtn]{2}[0-9] + | Intron length and splice signal |
| | [acgtn]{2} | |

For example, the sequence alignment shown in Table 2 for SEQ ID NO: 9 (upper sequence) and SEQ ID NO: 10 (lower sequence) is represented as :6−ata:10+gtc:4*at:3, where :[0-9]+ represents an identical block, −ata represents a deletion, +gtc represents an insertion, and *at indicates that the reference base “a” is substituted with “t”.

TABLE 2

| SEQ ID NO: 9 (Upper) and | |
|---|---|
| SEQ ID NO: 10 (Lower) | CIGAR |
| CGATCGATAAATAGAGTAG---GAATAGCA | :6-ata:10+gtc:4*at:3 |
| \|\|\|\|\|\|\| \|\|\|\|\|\|\|\|\|\| \|\|\|\| \|\|\| | |
| CGATCG---AATAGAGTAGGTCGAATtGCA | |

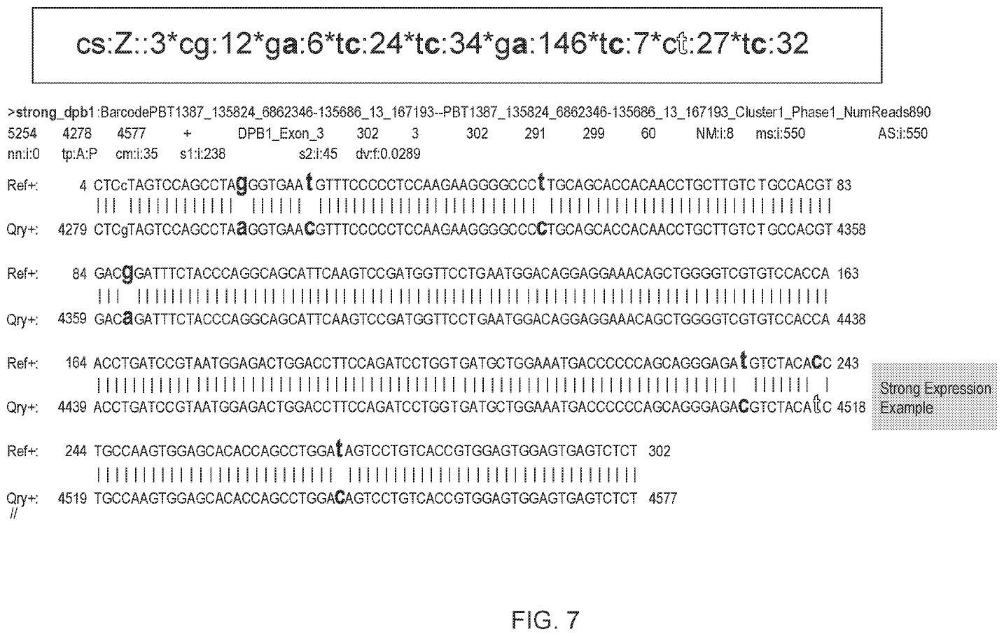

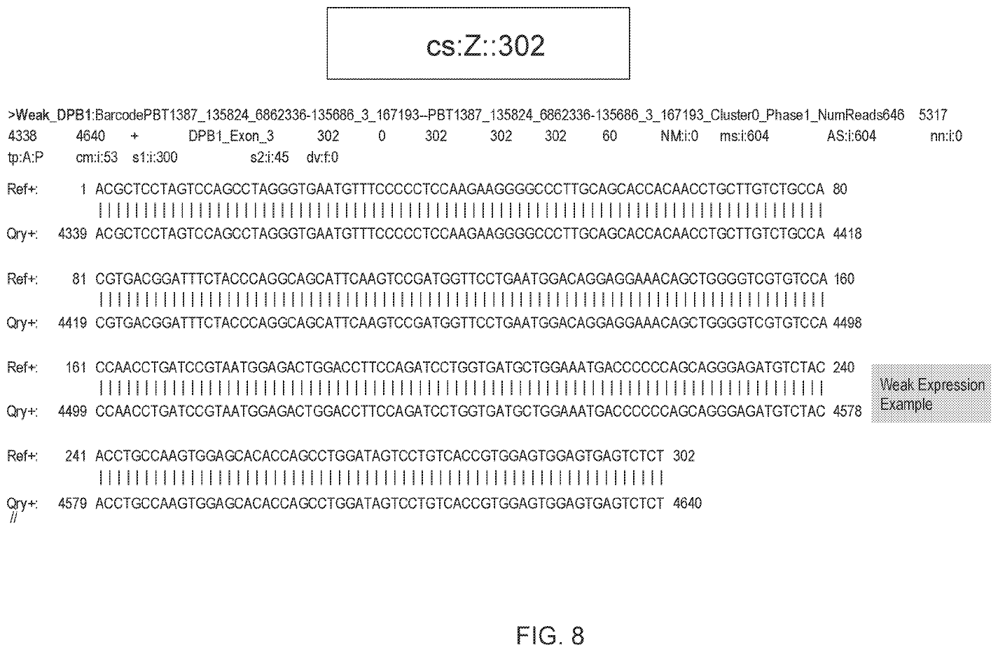

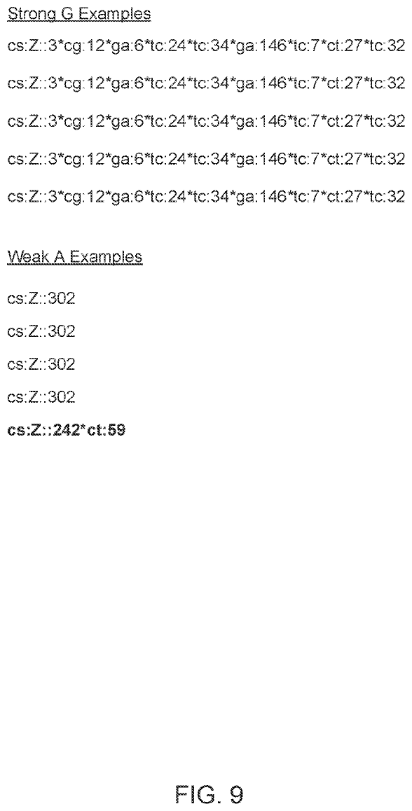

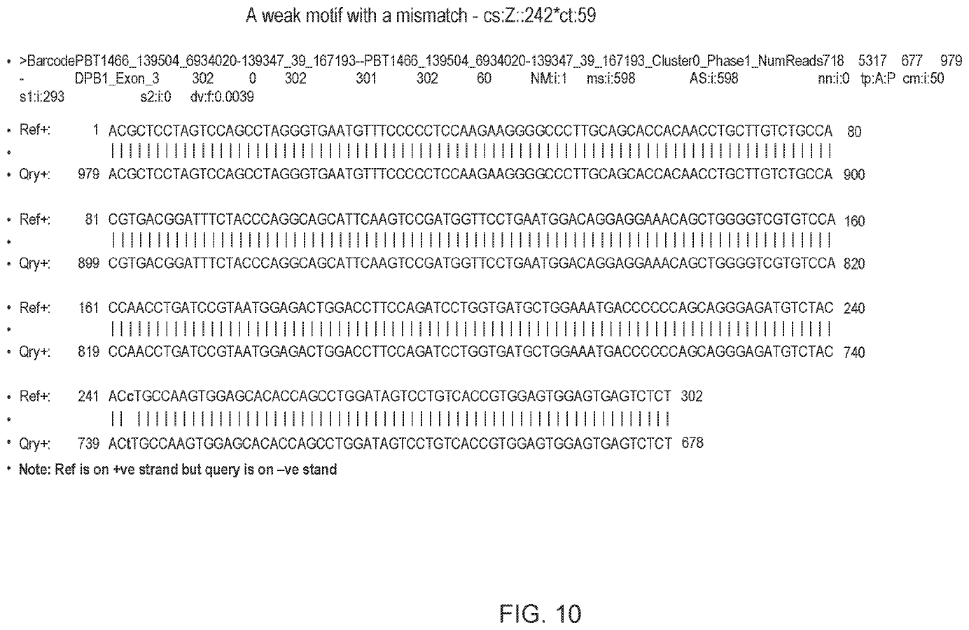

The algorithm can then parse the CIGAR to determine if the strong motif or the weak motif is present in the query sequence. Embodiments of strong and weak sequences reported using the CIGAR format are shown in FIGS. 7 and 8, respectively. Thus for the strong motif 3*cg:12*ga:6*tc:24*tc:34*ga:146*tc:7*ct:27*tc:32 (FIG. 7), the CIGAR coding is as follows: 3—number of bases that contig matches the reference before a variant; starts at the beginning of the reference; cg—variant is g instead of c; 12—number of bases the contig matches before the next variant; ga variant is a instead of g, and so on. The final number (“32”) is the number of bases that the contig matches the reference before the end of the reference. For the weak motif of FIG. 8 as compared to a weak variant, the CIGAR scoring is: cs:z::302 indicating that the query and the reference are identical over the 302 nucleotides of exon 3. In an embodiment, a sequence may comprise an overall match to either a weak motif or a long motif. An example of a weak motif with a mismatch at position 243 (i.e., one position removed from motif position 242) (cs:Z::242*ct:59) is shown in FIGS. 9 and 10.

### Systems for Analyzing Long Read Sequence Data to Predict DPB1 Expression Level

Embodiments of the disclosure include computerized systems and computer program products for analyzing long read sequence data to predict DPB1 expression level.

For example, disclosed is a system comprising one or more data processors, and a non-transitory computer readable storage medium containing instructions which, when executed on the one or more data processors, cause the one or more data processors to perform actions including analyzing sequence data from a subject to predict DPB1 expression level in the subject comprising the steps of: (a) obtaining, using a long read sequencer, a query nucleic acid sequence from a sample of the subject; (b) aligning, using a computer-implemented alignment program, the query nucleic acid sequence from the subject to a reference nucleic acid sequence, the reference and query nucleic acid sequences comprising long read sequence data for at least exon 3 of DPB1; and (c) identifying, using a computer implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises: (i) comparing nucleotides within the aligned query nucleic acid sequence and the reference nucleic acid sequence to identify differences between the query sequence nucleic acid sequence and the reference nucleic acid sequence; (ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1; (iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and (iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.

Also disclosed is a computer-program product tangibly embodied in a non-transitory machine-readable storage medium, including instructions configured to cause one or more data processors to perform actions including analyzing sequence data from a subject to predict DPB1 expression level in the subject comprising the steps of: (a) obtaining, using a long read sequencer, a query nucleic acid sequence from a sample of the subject; (b) aligning, using a computer-implemented alignment program, the query nucleic acid sequence from the subject to a reference nucleic acid sequence, the reference and query nucleic acid sequences comprising long read sequence data for at least exon 3 of DPB1; and (c) identifying, using a computer implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises: (i) comparing nucleotides within the aligned query nucleic acid sequence and the reference nucleic acid sequence to identify differences between the query sequence nucleic acid sequence and the reference nucleic acid sequence; (ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1; (iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and (iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.

The systems and computer products may perform any of the methods disclosed herein. One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

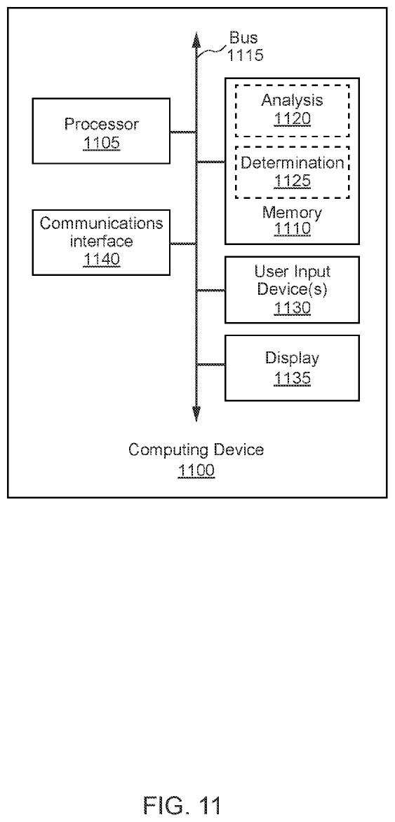

FIG. 11 shows a block diagram of a DPB1 sequence analysis system. As illustrated in FIG. 11, modules, engines, or components (e.g., program, code, or instructions) executable by one or more processors that may be used to implement the various subsystems of an analyzer system according to various embodiments. The modules, engines, or components may be stored on a non-transitory computer medium. As needed, one or more of the modules, engines, or components may be loaded into system memory (e.g., RAM) and executed by one or more processors of the analyzer system. In the example depicted in FIG. 11, modules, engines, or components are shown for implementing the methods of the disclosure.

Thus, FIG. 11 illustrates an example computing device 1100 suitable for use with systems and the methods according to this disclosure. The example computing device 1100 includes a processor 1105 which is in communication with the memory 1110 and other components of the computing device 1100 using one or more communications buses 1115. The processor 1105 is configured to execute processor-executable instructions stored in the memory 1110 to perform one or more methods for assessing DPB1 expression levels according to different examples, such as part or all of the example processes 500 or 600 described above with respect to FIGS. 5 and 6. In this example, the memory 1110 stores processor-executable instructions that provide DPB1 sequence analysis 1120 and expression determination 1125, as discussed herein.

The computing device 1100 in this example also includes one or more user input devices 1130, such as a keyboard, mouse, touchscreen, microphone, etc., to accept user input. The computing device 1100 also includes a display 1135 to provide visual output to a user such as a user interface. The computing device 1100 also includes a communications interface 1140. In some examples, the communications interface 1140 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### EXAMPLES

### Example 1—Identification of Strong and Weak Motifs

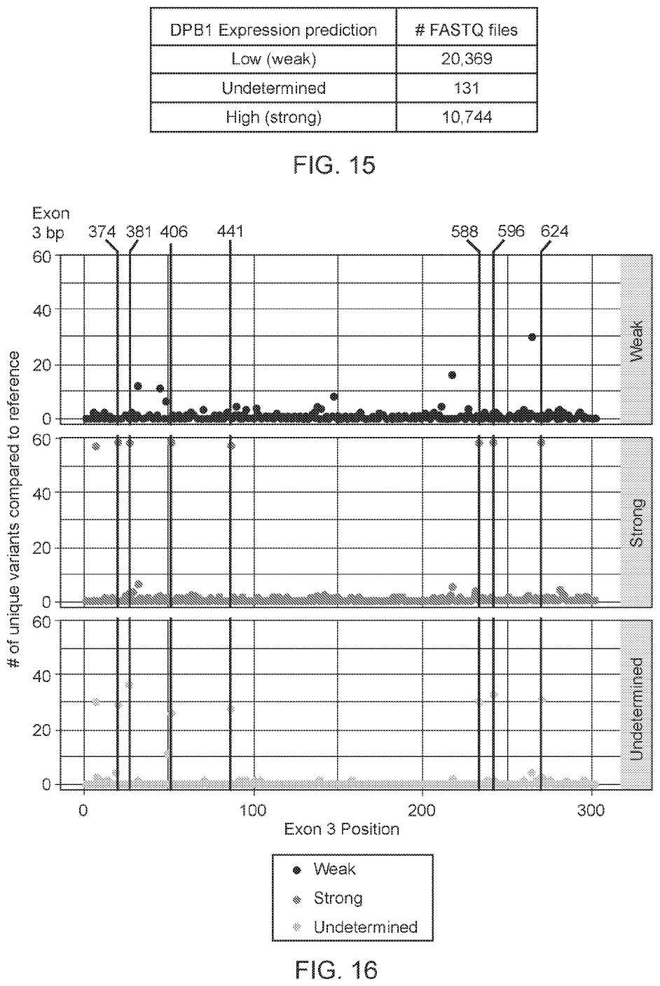

The disclosed methods have been used to characterize deidentified DNA sequences (i.e., sequences for which personal, registry or other identifying information has been removed such that sequences are identified by a random number) from a compilation of HLA typing production data for marrow donor registries. Sequence data from 17,801 previously typed individuals, stored in one FASTQ file per individual, were analyzed with this program. All FASTQ files that contained the exact seven base sequence that predicts a G at rs9277534 were typed as having a “Strong” expression. Then, all FASTQ files containing the exact seven base sequence that predicts an A at rs9277534 were typed as having a “Weak” expression. Any sequence that did not exactly match either motif was typed as “Undetermined.” As shown in FIG. 15, a total of 31,274 DPB1 sequences were analyzed and of those, 10,774 were typed as “Strong,” 20,369 were typed as “Weak,” and 131 were typed as “Undetermined.” 30 (0.001%) failed quality score criteria. Only 0.42% of all HLA-DPB1 sequences tested were ruled “Undetermined” while the remaining 99.58% exhibited the exact sequence necessary to predict expression. In addition, other variants were noted. 30 (0.001%) failed quality score criteria. All sequences were mapped to exon3 weak reference. The plot in FIG. 16 is a down sample of ˜60 samples per strong, weak and undetermined, and shows the positions in exon 3 where unique variants to the reference occur. The vertical lines shows the predictive positions.

Results are shown in FIG. 3 for a sequence having the strong motif. The query (Qry+) sequence (SEQ ID NO: 4) was found to have a DPB1 strong expression motif exon 3 sequence (solid line rectangles) at positions 20, 27, 52, 87, 234, 242 and 270, Further characterization of the sequence indicated that the query had other variants as compared to the reference at other locations in the exon (dashed line rectangles). In this figure, the sequencing starts at the fourth nucleotide for exon 3, and the reference sequence (Ref+) (SEQ ID NO: 3) has the weak expression motif.

FIG. 4 shows use of the disclosed algorithm to identify exon 3 motifs; the query (Qry+) sequence has a sequence of the weak expression motif (solid line rectangles) at positions 20, 27, 52, 87, 234, 242 and 270 as well as other variations (dashed line rectangles). In this figure, the sequencing starts at the fourth nucleotide for exon 3. In this experiment, the reference sequence (Ref+) has the weak expression motif.

FIGS. 7 and 8 show similar experiments performed with different query sequences and a reference sequence (SEQ ID NO: 3) having the exon 3 weak motif. As shown in FIG. 7, the Query sequence (SEQ ID NO: 6) displays the variants characteristic of the strong motif and is reported with the CIGAR notation as cs:Z::38cg:12*ga:6*tc:24*tc:34*ga:146*tc:7*ct:27*tc:32. There are no additional variants. Interestingly, this and other sequence analysis confirmed that the variant at position 242 is a T and not an A as originally reported (Schone et al., 2018). The query sequence shown in FIG. 8 (SEQ ID NO: 7) was defined as having weak expression based on its identity with the reference sequence (SEQ ID NO: 3) which contained the “weak expression” motif, and can be reported as cs:Z::302.

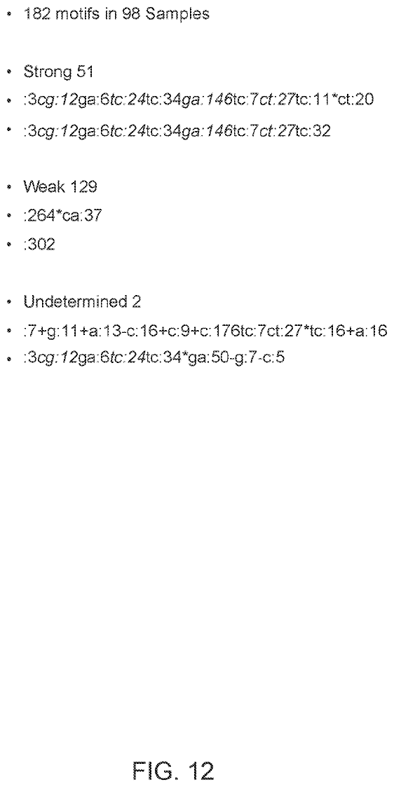

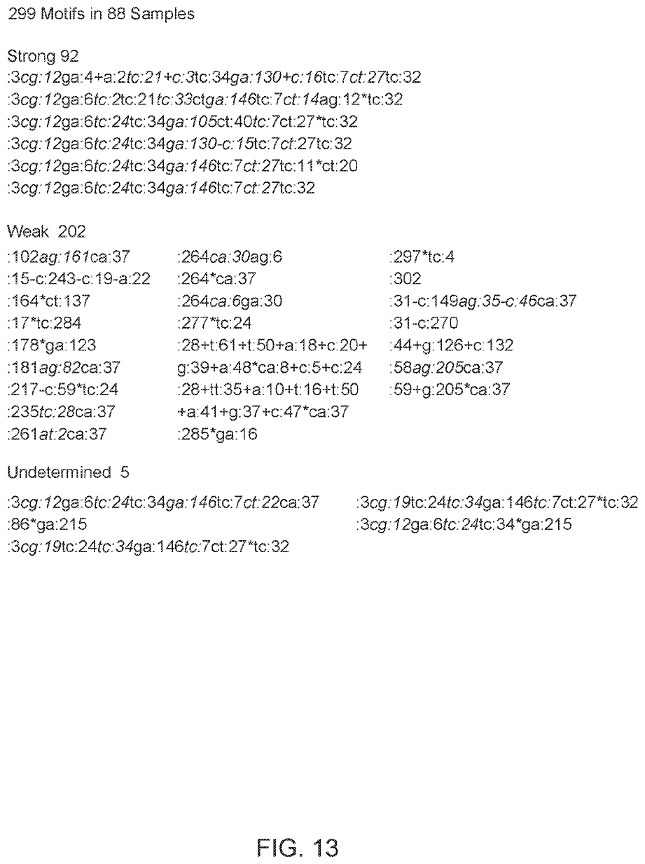

FIG. 9 presents a summary in CIGAR format for five samples characterized as having the strong expression motif and five samples having a “weak” expression motif. One of the weak expression samples has a T mutation at cDNA position 243; this is shown as the query (Qry−) sequence in FIG. 10 (SEQ ID NO: 8). Additional sequence results showing sequences having strong motifs (with and without additional variants) and weak motifs (with and without additional variants) as well as sequences classified as undetermined are shown in FIGS. 12 and 13. This sequence data may then be compiled and analyzed such that information regarding DPB1 expression can be evaluated in conjunction with other HLA typing data to pair potential hematopoietic stem cell donors and recipients.

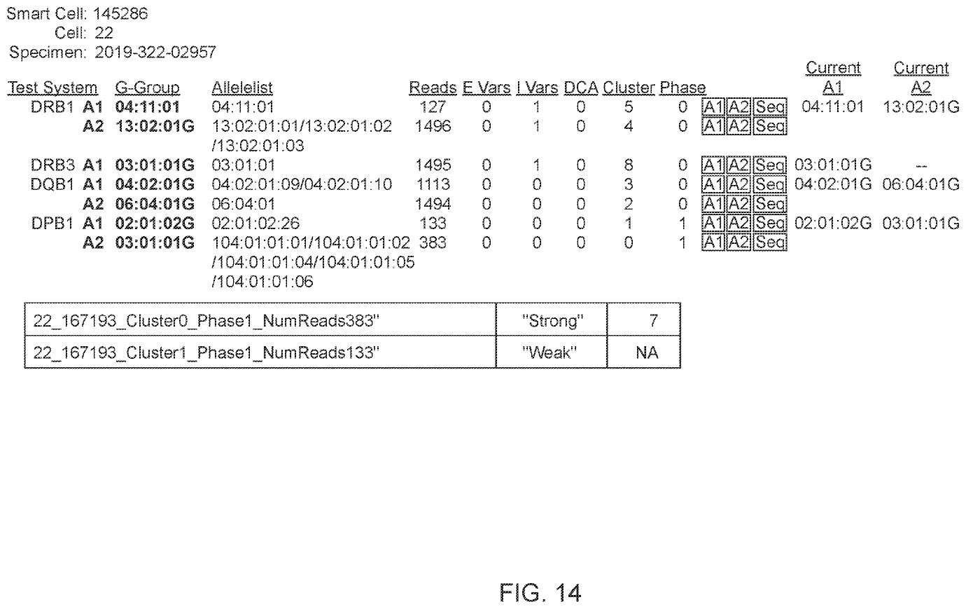

A print-out of the analysis of a sequence is shown in FIG. 14.

In summary, the data indicates that the disclosed HLA-DPB1 expression prediction program can be a very useful tool to aid in the selection of a donor for a patient in need of a hematopoietic stem cell transplant.

### Example 2—Embodiments

A1. A method for analyzing sequence data from a subject to predict DPB1 expression level in the subject comprising:

(a) aligning a query sequence from the subject to a reference sequence, the reference and query sequences comprising at least exon 3 of DPB1;

(b) comparing differences between the query sequence and the reference sequence;

(c) determining the identity of the nucleotides for the query sequence at defined positions in exon 3 of DPB1; and

(d) determining if the query exhibits the sequence characteristic of a weak expression motif or a strong expression motif or neither, thereby assessing the expression level of the subject's DPB1.

A2. The method of any of the previous or subsequent embodiments, wherein at least one of steps (a)-(d) is computer implemented.
A3. A computer-implemented method for analyzing sequence data from a subject to predict DPB1 expression level in the subject comprising the computer-implemented steps of:

(a) obtaining, using a long read sequencer, a query nucleic acid sequence from a sample of the subject;

(b) aligning, using a computer-implemented alignment program, the query nucleic acid sequence from the subject to a reference nucleic acid sequence, the reference and query nucleic acid sequences comprising long read sequence data for at least exon 3 of DPB1; and

(c) identifying, using a computer-implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises the following steps:
(i) comparing nucleotides within the aligned query nucleic acid sequence and the reference nucleic acid sequence to identify differences between the query nucleic acid sequence and the reference nucleic acid sequence;
(ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1;
(iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and
(iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.
   A4. The method of any of the previous or subsequent embodiments, wherein the defined positions of exon 3 are at positions 20, 27, 52, 87, 234, 242 and 270 from the 5′ end of the exon.
   A5. The method of any of the previous or subsequent embodiments, further comprising defining the weak expression motif as comprising: a G at position 20 of exon 3, a T at position 27 of exon 3, a T at position 52 of exon 3, a G at position 87 of exon 3, a T at position 234 of exon 3, a C at position 242 of exon 3, and a Tat position 270 of exon 3.
   A6. The method of any of the previous or subsequent embodiments, further comprising defining the strong expression motif as comprising: an A at position 20 of exon 3, a C at position 27 of exon 3, a C at position 52 of exon 3, an A at position 87 of exon 3, a C at position 234 of exon 3, a Tat position 242 of exon 3, and a C at position 270 of exon 3.
   A7. The method of any of the previous or subsequent embodiments, wherein if the nucleotides at exon 3 positions 20, 27, 52, 87, 234, 242 and 270 are not characteristic of either the weak expression motif or the strong expression motif the allele is identified as indeterminate.
   A8. The method of any of the previous or subsequent embodiments, wherein the identifying further comprises comparing nucleotides within the aligned query nucleic acid sequence and the reference nucleic acid sequence to identify the existence of any differences between the query nucleic acid sequence and the reference nucleic acid sequence at positions of exon 3 other than the defined positions.
   A9. The method of any of the previous or subsequent embodiments, wherein the identifying further comprises comparing nucleotides within the aligned query nucleic acid sequence and the reference nucleic acid sequence to identify the existence of any other differences between the query nucleic acid sequence and the reference nucleic acid sequence.
   A10. The method of any of the previous or subsequent embodiments, wherein if the number of differences between the query nucleic acid sequence and the reference nucleic acid sequence at positions other than the defined positions in exon 3 is greater than ten, excluding the query sequence from further analysis.
   A11. The method of any of the previous or subsequent embodiments, further comprising determining, optionally by computer-implemented linkage analysis, that the strong expression motif in the query nucleic acid sequence is linked to a rs9277534 G allele and/or that the weak expression motif in the query nucleic acid sequence is linked to the rs9277534 A allele.
   A12. The method of any of the previous or subsequent embodiments, wherein the reference and/or query nucleic acid sequence comprises long read sequence data for the entire DPB1 gene.
   A13. The method of any of the previous or subsequent embodiments, wherein the reference and/or query nucleic acid sequence further comprises long read sequence data for at least one of DRB1, DRB3, DQB1, DRB4, DRB5, DQA1, or DPA1.
   A14. The method of any of the previous or subsequent embodiments, further comprising providing the results to a caregiver and/or a transplant database to reduce a risk of graft vs host disease in a transplant recipient.
   A15. The method of any of the previous or subsequent embodiments, wherein if the number of differences between the query nucleic acid sequence and the reference nucleic acid sequence at positions other than the defined positions in exon 3 is greater than ten, the results for the query sequence are not provided to a caregiver or a database.
   A16. The method of any of the previous or subsequent embodiments, wherein the subject is a potential donor for a hematopoietic stem cell transplant (HSCT) recipient.
   A17. The method of any of the previous or subsequent embodiments, further comprising the step of determining the query nucleic acid sequence for the subject.
   A18. The method of any of the previous or subsequent embodiments, wherein the query sequence is obtained from long read or short read sequence data generated (prior to step (a)) as part of a next generation sequencing experiment.
   B1. A system comprising:

one or more data processors; and

a non-transitory computer readable storage medium containing instructions which, when executed on the one or more data processors, cause the one or more data processors to perform actions including analyzing sequence data from a subject to predict DPB1 expression level in the subject comprising the steps of:

(a) aligning a query nucleic acid sequence from the subject to a reference nucleic acid sequence, the reference sequence and the query sequence comprising at least exon 3 of DPB1;

(b) comparing differences between the query sequence and the reference sequence;

(c) determining the identity of the nucleotides for the query sequence at defined positions in exon 3 of DPB1; and

(d) determining if the query exhibits the sequence characteristic of a weak expression motif or a strong expression motif or neither, thereby assessing the expression level of the subject's DPB1.

B2. A system comprising:

one or more data processors; and

a non-transitory computer readable storage medium containing instructions which, when executed on the one or more data processors, cause the one or more data processors to perform actions including analyzing sequence data from a subject to predict DPB1 expression level in the subject comprising the computer-implemented steps of:

(a) obtaining, using a long read sequencer, a query nucleic acid sequence from a sample of the subject;

(b) aligning, using a computer-implemented alignment program, the query nucleic acid sequence from the subject to a reference nucleic acid sequence, the reference and query nucleic acid sequences comprising long read sequence data for at least exon 3 of DPB1; and

(c) identifying, using a computer-implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises the following steps:
(i) comparing nucleotides within the aligned query nucleic acid sequence and the reference nucleic acid sequence to identify differences between the query nucleic acid sequence and the reference nucleic acid sequence;
(ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1;
(iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and
(iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.
   B3. The system of any of the previous or subsequent embodiments, wherein the actions further include defining the weak expression motif as comprising: a G at position 20 of exon 3, a T at position 27 of exon 3, a T at position 52 of exon 3, a G at position 87 of exon 3, a T at position 234 of exon 3, a C at position 242 of exon 3, and a T at position 270 of exon 3.
   B4. The system of any of the previous or subsequent embodiments, wherein the actions further include defining the strong expression motif as comprising: an A at position 20 of exon 3, a C at position 27 of exon 3, a C at position 52 of exon 3, an A at position 87 of exon 3, a C at position 234 of exon 3, a T at position 242 of exon 3, and a C at position 270 of exon 3.
   B5. The system of any of the previous or subsequent embodiments, wherein if the nucleotides at exon 3 positions 20, 27, 52, 87, 234, 242 and 270 are not characteristic of either the weak expression motif or the strong expression motif, the allele is defined as indeterminate.
   B6. The system of any of the previous or subsequent embodiments, wherein the identifying further comprises comparing nucleotides within the aligned query nucleic acid sequence and the reference nucleic acid sequence to identify the existence of any differences between the query nucleic acid sequence and the reference nucleic acid sequence at positions of exon 3 other than the defined positions.
   B7. The system of any of the previous or subsequent embodiments, wherein the identifying further comprises comparing nucleotides within the aligned query nucleic acid sequence and the reference nucleic acid sequence to identify the existence of any other differences between the query nucleic acid sequence and the reference nucleic acid sequence.
   B8. The system of any of the previous or subsequent embodiments, wherein if the number of differences between the query nucleic acid sequence and the reference nucleic acid sequence at positions other than the defined positions in exon 3 is greater than ten, excluding the query sequence from further analysis.
   B9. The system of any of the previous or subsequent embodiments, wherein the actions further comprises determining that the strong expression motif is linked to the rs9277534 G allele or the weak expression motif is linked to the rs9277534 A allele.
   B10. The system of any of the previous or subsequent embodiments, wherein the actions further comprise determining, optionally by computer-implemented linkage analysis, that the strong expression motif in the query nucleic acid sequence is linked to a rs9277534 G allele and/or that the weak expression motif in the query nucleic acid sequence is linked to the rs9277534 A allele.
   B11. The system of any of the previous or subsequent embodiments, wherein the reference and/or query sequence comprises long read sequence data for the entire DPB1 gene.
   B12. The system of any of the previous or subsequent embodiments, wherein the reference and/or query sequence further comprises long read sequence data for at least one of DRB1, DRB3, DQB1, DRB1, DRB3, DQB1, DRB4, DRB5, DQA1, or DPA1.
   B13. The system of any of the previous or subsequent embodiments, wherein the actions further comprise providing the results to a caregiver and/or a database to reduce the risk of graft vs host disease in a transplant recipient.
   B14. The system of any of the previous or subsequent embodiments, wherein if the number of differences between the query nucleic acid sequence and the reference nucleic acid sequence at positions other than the defined positions in exon 3 is greater than ten, the results for the query sequence are not provided to a caregiver or a database.
   B15. The system of any of the previous or subsequent embodiments, wherein the subject is a potential donor for a hematopoietic stem cell transplant (HSCT) recipient.
   B16. The system of any of the previous or subsequent embodiments, wherein at least one of steps (a)-(d) is computer implemented.
   B17. The system of any of the previous or subsequent embodiments, wherein the actions further comprise the step of determining the query sequence for the subject.
   B18. The system of any of the previous or subsequent embodiments, wherein the query sequence is obtained from long read or short read sequence data generated (prior to step (a)) as part of a sequencing experiment.
   B19. The system of B18, wherein the sequencing experiment is a next generation sequencing experiment.
   B20. A system comprising:

one or more data processors; and

a non-transitory computer readable storage medium containing instructions which, when executed on the one or more data processors, cause the one or more data processors to perform actions for performing the methods of any of the previous embodiments.

C1. A computer-program product tangibly embodied in a non-transitory machine-readable storage medium, including instructions configured to run the systems and/or perform the methods of any of the previous or subsequent embodiments.
C2 A computer-program product tangibly embodied in a non-transitory machine-readable storage medium, including instructions configured to cause one or more data processors to perform actions including analyzing sequence data from a subject to predict DPB1 expression level in the subject comprising the steps of:

(a) obtaining, using a long read sequencer, a query nucleic acid sequence from a sample of the subject;

(b) aligning, using a computer-implemented alignment program, the query nucleic acid sequence from the subject to a reference nucleic acid sequence, the reference and query nucleic acid sequences comprising long read sequence data for at least exon 3 of DPB1; and

(c) identifying, using a computer-implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises the following steps:
(i) comparing nucleotides within the aligned query nucleic acid sequence and the reference nucleic acid sequence to identify differences between the query nucleic acid sequence and the reference nucleic acid sequence;
(ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1;
(iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and
(iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.
   C3. The computer program product of any of the previous or subsequent embodiments, wherein the actions further include defining the weak expression motif as comprising: a G at position 20 of exon 3, a T at position 27 of exon 3, a T at position 52 of exon 3, a G at position 87 of exon 3, a T at position 234 of exon 3, a C at position 242 of exon 3, and a T at position 270 of exon 3.
   C4. The computer program product of any of the previous or subsequent embodiments, wherein the actions further include defining the strong expression motif as comprising: an A at position 20 of exon 3, a C at position 27 of exon 3, a C at position 52 of exon 3, an A at position 87 of exon 3, a C at position 234 of exon 3, a T at position 242 of exon 3, and a C at position 270 of exon 3.
   C5. The computer program product of any of the previous or subsequent embodiments, wherein the identifying further comprises comparing nucleotides within the aligned query nucleic acid sequence and the reference nucleic acid sequence to identify the existence of any differences between the query nucleic acid sequence and the reference nucleic acid sequence at positions of exon 3 other than the defined positions.
   C6. The computer program product of any of the previous or subsequent embodiments, wherein the identifying further comprises comparing nucleotides within the aligned query nucleic acid sequence and the reference nucleic acid sequence to identify the existence of any other differences between the query nucleic acid sequence and the reference nucleic acid sequence.
   C7. The computer program product of any of the previous or subsequent embodiments, wherein if the number of differences between the query nucleic acid sequence and the reference nucleic acid sequence at positions other than the defined positions in exon 3 is greater than ten, excluding the query sequence from further analysis.
   C8. The computer program product of any of the previous or subsequent embodiments, wherein the actions further comprises determining that the strong expression motif is linked to the rs9277534 G allele or the weak expression motif is linked to the rs9277534 A allele.
   C9. The computer program product of any of the previous or subsequent embodiments, wherein the actions further comprise determining, optionally by computer-implemented linkage analysis, that the strong expression motif in the query nucleic acid sequence is linked to a rs9277534 G allele and/or that the weak expression motif in the query nucleic acid sequence is linked to the rs9277534 A allele.
   C10. The computer program product of any of the previous or subsequent embodiments, wherein the reference and/or query sequence comprises long read sequence data for the entire DPB1 gene.
   C11. The computer program product of any of the previous or subsequent embodiments, wherein the reference and/or query sequence further comprises long read sequence data for at least one of DRB1, DRB3, DQB1, DRB1, DRB3, DQB1, DRB4, DRB5, DQA1, or DPA1.
   C12. The computer program product of any of the previous or subsequent embodiments, wherein the actions further comprise providing the results to a caregiver and/or a database to reduce the risk of graft vs host disease in a transplant recipient.
   C13. The computer program product of any of the previous or subsequent embodiments, wherein if the number of differences between the query nucleic acid sequence and the reference nucleic acid sequence at positions other than the defined positions in exon 3 is greater than ten, the results for the query sequence are not provided to a caregiver or a database.
   C14. The computer program product of any of the previous or subsequent embodiments, wherein the subject is a potential donor for a hematopoietic stem cell transplant (HSCT) recipient.
   C15. The computer program product of any of the previous or subsequent embodiments, wherein at least one of steps (a)-(d) is computer implemented.
   C16. The computer program product of any of the previous or subsequent embodiments, wherein the actions further comprise the step of determining the query nucleic acid sequence for the subject.
   C17. The computer program product of any of the previous or subsequent embodiments, wherein the query sequence is obtained from long read or short read sequence data generated (prior to step (a)) as part of a sequencing experiment.
   C18. The computer program product of any of the previous or subsequent embodiments, wherein the sequencing experiment is a next generation sequencing experiment.

### Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein.

Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

1. A computer-implemented method for analyzing long read sequence data from a subject to predict DPB1 expression level in the subject comprising the computer-implemented steps of:
(a) obtaining, using a long read sequencer, a query nucleic acid sequence from a sample of the subject;
(b) aligning, using a computer-implemented alignment program, the query nucleic acid sequence from the subject to a reference nucleic acid sequence, the reference and query nucleic acid sequences comprising long read sequence data for at least exon 3 of DPB1; and
(c) identifying, using a computer-implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises the following steps:
(i) comparing nucleotides within the aligned query nucleic acid sequence and the reference nucleic acid sequence to identify differences between the query nucleic acid sequence and the reference nucleic acid sequence;
(ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1;
(iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and
(iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.

2. The method of claim 1, wherein the defined positions of exon 3 are at positions 20, 27, 52, 87, 234, 242 and 270 from the 5′ end of the exon.

3. The method of claim 1, further comprising defining the weak expression motif as comprising: a G at position 20 of exon 3, a Tat position 27 of exon 3, a T at position 52 of exon 3, a G at position 87 of exon 3, a T at position 234 of exon 3, a C at position 242 of exon 3, and a T at position 270 of exon 3.

4. The method of claim 1, further comprising defining the strong expression motif as comprising: an A at position 20 of exon 3, a C at position 27 of exon 3, a C at position 52 of exon 3, an A at position 87 of exon 3, a C at position 234 of exon 3, a T at position 242 of exon 3, and a C at position 270 of exon 3.

5. The method of claim 2, wherein if the nucleotides at exon 3 positions 20, 27, 52, 87, 234, 242 and 270 are not characteristic of either the weak expression motif or the strong expression motif the allele is identified as indeterminate.

6. The method of claim 1, wherein the identifying further comprises comparing nucleotides within the aligned query nucleic acid sequence and the reference nucleic acid sequence to identify the existence of any differences between the query nucleic acid sequence and the reference nucleic acid sequence at positions of exon 3 other than the defined positions.

7. The method of claim 6, wherein the identifying further comprises comparing nucleotides within the aligned query nucleic acid sequence and the reference nucleic acid sequence to identify the existence of any other differences between the query nucleic acid sequence and the reference nucleic acid sequence.

8. The method of claim 1, wherein if the number of differences between the query nucleic acid sequence and the reference nucleic acid sequence at positions other than the defined positions in exon 3 is greater than ten, excluding the query sequence from further analysis.

9. The method of claim 1, further comprising performing computer-implemented linkage analysis to determine that the strong expression motif in the query nucleic acid sequence is linked to a rs9277534 G allele and/or that the weak expression motif in the query nucleic acid sequence is linked to the rs9277534 A allele.

10. The method of claim 1, wherein the reference and/or query nucleic acid sequence comprises long read sequence data for the entire DPB1 gene.

11. The method of claim 9, wherein the reference and/or query nucleic acid sequence further comprises long read sequence data for at least one of DRB1, DRB3 and DQB1.

12. The method of claim 1, further comprising providing the results to a caregiver and/or a transplant database to reduce a risk of graft vs host disease in a transplant recipient.

13. The method of claim 12, wherein if the number of differences between the query nucleic acid sequence and the reference nucleic acid sequence at positions other than the defined positions in exon 3 is greater than ten, the results for the query sequence are not provided to a caregiver or a database.

14. The method of claim 1, wherein the subject is a potential donor for a hematopoietic stem cell transplant (HSCT) recipient.

15. A system comprising:
one or more data processors; and
a non-transitory computer readable storage medium containing instructions which, when executed on the one or more data processors, cause the one or more data processors to perform actions including analyzing sequence data from a subject to predict DPB1 expression level in the subject comprising the computer-implemented steps of:
(a) obtaining, using a long read sequencer, a query nucleic acid sequence from a sample of the subject;
(b) aligning, using a computer-implemented alignment program, the query nucleic acid sequence from the subject to a reference nucleic acid sequence, the reference and query nucleic acid sequences comprising long read sequence data for at least exon 3 of DPB1; and
(c) identifying, using a computer-implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises the following steps:
(i) comparing nucleotides within the aligned query nucleic acid sequence and the reference nucleic acid sequence to identify differences between the query nucleic acid sequence and the reference nucleic acid sequence;
(ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1;
(iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and
(iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.

16. The system of claim 15, wherein the actions further include defining the weak expression motif as comprising: a G at position 20 of exon 3, a T at position 27 of exon 3, a T at position 52 of exon 3, a G at position 87 of exon 3, a T at position 234 of exon 3, a C at position 242 of exon 3, and a Tat position 270 of exon 3.

17. The system of claim 15, wherein the actions further include defining the strong expression motif as comprising: an A at position 20 of exon 3, a C at position 27 of exon 3, a C at position 52 of exon 3, an A at position 87 of exon 3, a C at position 234 of exon 3, a T at position 242 of exon 3, and a C at position 270 of exon 3.

18. A computer-program product tangibly embodied in a non-transitory machine-readable storage medium, including instructions configured to cause one or more data processors to perform actions including analyzing sequence data from a subject to predict DPB1 expression level in the subject comprising the computer-implemented steps of:
(a) obtaining, using a long read sequencer, a query nucleic acid sequence from a sample of the subject;
(b) aligning, using a computer-implemented alignment program, the query nucleic acid sequence from the subject to a reference nucleic acid sequence, the reference and query nucleic acid sequences comprising long read sequence data for at least exon 3 of DPB1; and
(b) identifying, using a computer-implemented algorithm, whether the query nucleic acid sequence has a sequence characteristic of low levels of DPB1 expression or high levels of DPBI expression based on the aligned query nucleic acid sequence and the reference nucleic acid sequence, wherein the identifying comprises the following steps:
(i) comparing nucleotides within the aligned query nucleic acid sequence and the reference nucleic acid sequence to identify differences between the query nucleic acid sequence and the reference nucleic acid sequence;
(ii) determining, based on the identified differences between the query sequence nucleic acid sequence and the reference nucleic acid sequence, an identity of the nucleotides for the query nucleic acid sequence as compared to the reference nucleic acid sequence at defined positions in the exon 3 of DPB1;
(iii) determining, based on the identity of the nucleotides for the query nucleic acid sequence at the defined positions in the exon 3, that the query sequence exhibits a sequence characteristic of a weak expression motif or a sequence characteristic of a strong expression motif or neither; and
(iv) identifying the subject as having a low expression level of DPB1 when the query nucleic acid sequence exhibits the sequence characteristic of the weak expression motif, or identifying the subject as having a high expression level of DPB1 when the query nucleic acid sequence exhibits the sequence characteristic of the strong expression motif.

19. The computer program product of claim 18, wherein the actions further include defining the weak expression motif as comprising: a G at position 20 of exon 3, a T at position 27 of exon 3, a T at position 52 of exon 3, a G at position 87 of exon 3, a T at position 234 of exon 3, a C at position 242 of exon 3, and a T at position 270 of exon 3.

20. The computer program product of claim 18, wherein the actions further include defining the strong expression motif as comprising: an A at position 20 of exon 3, a C at position 27 of exon 3, a C at position 52 of exon 3, an A at position 87 of exon 3, a C at position 234 of exon 3, a Tat position 242 of exon 3, and a C at position 270 of exon 3.
